(19) United States
(12) Patent Application Publication
(10) Pub. No.: US 2009/0035224 A1
(43) Pub. Date: Feb. 5, 2009

(54) Methods of Using Albuterol and Calcium Activated Potassium Channel Openers

(75) Inventors: Anne M. Sullivan, Boston, MA (US); John W. Simon, Westborough, MA (US); Yael Schwartz, Worcester, MA (US)

Correspondence Address: JONES DAY, 222 EAST 41ST ST, NEW YORK, NY, 10017, US

(73) Assignee: SEPRACOR INC., Marlborough, MA (US)

(21) Appl. No.: 11/629,686

(22) PCT Filed: Jun. 14, 2005
(86) PCT No.: PCT/US05/21171

§ 371 Date: Sep. 4, 2008

Related U.S. Application Data
(60) Provisional application No. 60/578,827, filed on Jun. 14, 2004.
(60) Provisional application No. 60/599,027, filed on Aug. 6, 2004.
(60) Provisional application No. 60/607,290, filed on Sep. 7, 2004.

(51) Int. Cl. A61K 31/137; A61K 31/41; A61K 31/704; A61P 11/00

(52) U.S. Cl. 424/43; 514/653; 514/381; 514/34

(57) ABSTRACT

This invention related to methods of treating, preventing and managing various pulmonary or respiratory diseases or disorders using albuterol in combination with calcium activated potassium channel openers. Pharmaceuticals compositions comprising albuterol and calcium activated potassium channel openers are also disclosed.

### 1. FIELD OF THE INVENTION

This invention relates to methods and compositions for treating, preventing and/or managing of various pulmonary or respiratory diseases and disorders.

### 2. BACKGROUND OF THE INVENTION

Albuterol is a drug belonging to the general class of beta-adrenergic receptor antagonists. The prime action of beta-adrenergic drugs is to stimulate adenyl cyclase, the enzyme which catalyzes the formation of cyclic-3′,5′-adenosine monophosphate (AMP) from adenosine triphosphate (ATP). The cyclic AMP formed mediates the cellular responses. Albuterol acts on β₂-adrenergic receptors to relax smooth muscle tissue, for example, in the respiratory system. Albuterol is most commonly used to treat bronchial smooth muscle spasms associated with asthma and is the active component in well-known commercial bronchodilators such as Proventil and Ventolin.

Although various therapies are currently practiced for the treatment of pulmonary diseases or disorders, many adverse effects have been associated with the conventional therapies. Moreover, the effectiveness of a single therapy in treating a pulmonary disease or disorder is still questionable. Therefore, an on-going need exists for an improved therapeutics for treating, preventing and/or managing pulmonary diseases or disorders with reduced adverse effects.

### 3. SUMMARY OF THE INVENTION

This invention encompasses a method of treating, preventing and/or managing pulmonary diseases or disorders, by administering to a patient a therapeutically or prophylactically effective amount of albuterol, or a pharmaceutically acceptable salt, solvate, stereoisomer, or prodrug thereof, in combination with a calcium activated potassium channel opener, or a pharmaceutically acceptable salt, solvate, or prodrug thereof.

In one embodiment, this invention encompasses a method of treating, preventing and/or managing pulmonary diseases or disorders, by administering to a patient a therapeutically or prophylactically effective amount of albuterol, or a pharmaceutically acceptable salt, solvate, stereoisomer, or prodrug thereof, in combination with a calcium activated potassium channel opener, or a pharmaceutically acceptable salt, solvate, or prodrug thereof, while reducing the adverse effects associated with conventional therapies for such diseases or disorders.

Methods of this invention provide a safe, effective method for treating, preventing and/or managing various pulmonary diseases or disorders, while reducing undesirable side effects, for example, tremor, nervousness, shakiness, dizziness and increased appetite, and cardiac arrhythmia. In children, side effects such as excitement, nervousness, and hyperkinesia are reduced.

This invention also encompasses pharmaceutical compositions comprising albuterol, or a pharmaceutically acceptable salt, solvate, stereoisomer, or prodrug thereof, and a calcium activated potassium channel opener, or a pharmaceutically acceptable salt, solvate, or prodrug thereof.

### 4. DETAILED DESCRIPTION OF THE INVENTION

This invention is based, in part, on a belief that albuterol can be combined with other pharmacological agents, such as calcium activated potassium channel openers, for the treatment, prevention or management of pulmonary diseases and disorders. Without being limited by theory, this combination is believed to be more effective, have fewer adverse effects, and/or provide an overall improved therapeutic index as compared to prior methods of treating pulmonary or respiratory diseases and disorders. It is further anticipated that the combination will reduce paradoxical bronchospasm.

4.1 Methods of Treatment, Prevention and Management

This invention encompasses methods of treating, preventing and managing pulmonary diseases or disorders comprising administering to a patient in need of such treatment, prevention or management a therapeutically or prophylactically effective amount of albuterol, or a pharmaceutically acceptable salt, solvate, stereoisomer, or prodrug thereof, and a therapeutically or prophylactically effective amount of a calcium activated potassium channel opener, or a pharmaceutically acceptable salt, solvate, or prodrug thereof.

This invention also encompasses methods of treating, preventing and managing pulmonary diseases or disorders comprising administering to a patient in need of such treatment, prevention or management a therapeutically or prophylactically effective amount of albuterol, or a pharmaceutically acceptable salt, solvate, stereoisomer, or prodrug thereof, and a therapeutically or prophylactically effective amount of a calcium activated potassium channel opener, or a pharmaceutically acceptable salt, solvate, or prodrug thereof, while avoiding or reducing adverse effects associated with conventional therapies for such diseases or disorders. Examples of adverse effects include, but are not limited to, central nervous system stimulatory effects and undesirable cardiac effects, such as, but not limited to, tremor, nervousness, shakiness, dizziness, increased appetite, and cardiac arrhythmia. In children, side effects such as excitement, nervousness, and hyperkinesia are reduced.

As used herein, and unless otherwise specified, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. Preferred are hydrochloric, hydrobromic, phosphoric, sulfuric and tataric acids. In one embodiment, preferred are sulfate salt and the free base. In another embodiment, preferred are hydrochloric and L-tartaric acid salts.

As used herein, and unless otherwise specified, the term “solvate” means a compound of the present invention or a salt thereof, that further includes a stoichiometric or non-stoichiometric amount of solvent bound by non-covalent intermolecular forces. Where the solvent is water, the solvate is a hydrate.

As used herein, and unless otherwise specified, the term “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, 95%, 97%, 99%, 99.5%, or 99.75% or more of one stereoisomer and 20%, 10%, 5%, 3%, 1%, 0.5%, or 0.25% or less of the counter stereoisomer. In certain cases, a compound of the invention is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses unequal mixtures of stereomerically pure isomers of compounds of this invention (e.g., R/S=30/70, 35/65, 40/60, 45/55, 55/45, 60/40, 65/35 and 70/30).

Furthermore, this invention also encompasses the use of equal (racemic) mixtures of stereomerically pure isomers of compounds of this invention.

As used herein, and unless otherwise specified, the term “prodrug” means a biologically active derivative of a compound that can hydrolyze, oxidize, or otherwise react under biological conditions (in vitro or in vivo) to provide the pharmacologically active compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties.

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder.

As used herein, unless otherwise specified, the terms “prevent,” “preventing” and “prevention” contemplate an action that occurs before a patient begins to suffer from the specified disease or disorder, which inhibits or reduces the severity of the disease or disorder.

As used herein, and unless otherwise specified, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

In one embodiment, albuterol, or a pharmaceutically acceptable salt, solvate, stereoisomer, or prodrug thereof, is used in combination with a calcium activated potassium channel opener, or a pharmaceutically acceptable salt, solvate, or prodrug thereof. Examples of calcium activated potassium channel openers include, but are not limited to, andolast (CR 2039), dehydrosoyasaponin I (DHS-I), soyasaponin I, soyasaponin III, NS 004, NS 1619, and BRL 55834.

In one embodiment, albuterol is racemic. In another embodiment, albuterol is stereomerically pure. In another embodiment, the stereomerically pure albuterol is R(−)-abluterol. In another embodiment, the stereomerically pure albuterol is S(+)-albuterol.

Andolast is chemically named N-4-(5-tetrazolyl)-phenyl-4-(5-tetrazolyl)-benzamide. Andolast and methods of making it are disclosed in U.S. Pat. No. 5,232,937, which is incorporated herein by reference. DHS-I, soyasaponin I, and soyasaponin III refer to triterpenes found in extracts from Desmodium adscendens. See Kaczorowski et al. in Airways Smooth Muscle: Peptide Receptors, Ion Channels and Signal Transduction (Basel: Birkhauser Verlag, 1995), p.p. 169-198, incorporated herein by reference. NS 004 and NS 1619 are benzimidazole derivatives that act as calcium activated potassium channel openers. See Macmillan et al., Brit. J. Pharmacol., 116: 1656-1660 (1995), incorporated herein by reference. BRL 55834 is described, for example, in Ward et al., Brit. J. Pharmacol., 107: 49P (1992), incorporated herein by reference.

In one embodiment, the calcium activated potassium channel opener is andolast. In another embodiment, the calcium activated potassium channel opener is DHS-I.

Various pulmonary or respiratory diseases or disorders can be treated, prevented and/or managed using methods of the invention. Examples of pulmonary or respiratory diseases or disorders include, but are not limited to: respiratory failure; adult respiratory distress syndrome; chronic obstructive airway disorders such as, but not limited to, asthma, chronic obstructive pulmonary disease and giant bullae; acute bronchitis; chronic bronchitis; emphysema; reversible obstructive airway disease; nocturnal asthma; exercise induced bronchospasm; long-term maintenance treatment of asthma; prevention of bronchospasm in patients with reversible obstructive airway disease, including patients with symptoms of asthma, who require treatment with other inhaled short-acting β₂-adrenergic agonists; long-term management of bronchoconstriction associated with chronic obstructive pulmonary disease, including chronic bronchitis and emphysema; acute prevent of exercise-induced bronchospasm, used in occasional, as needed, basis; bronchiectasis; bronchiolitis; cystic fibrosis; eatelectasis; pulmonary embolism; pneumonia; GERD; lung abscess; hypersensitivity of the lung such as, but not limited to, hypersensitivity pneumonitis, eosinophilic pneumonias and allergic bronchopulmonary aspergillosis; and Goodpasture's syndrome.

Albuterol, or a pharmaceutically acceptable salt, solvate, stereoisomer, or prodrug thereof, and a calcium activated potassium channel opener, or a pharmaceutically acceptable salt, solvate, hydrate, clathrate or prodrug thereof, can be administered sequentially or concurrently.

Albuterol, or a pharmaceutically acceptable salt, solvate, stereoisomer, or prodrug thereof, is preferably administered in an amount of from about 0.01 mg to about 1000 mg per day, from about 0.1 mg to about 300 mg per day, from about 1 mg to about 100 mg per day, or from about 1 mg to about 10 mg per day.

Suitable daily dosage ranges of the calcium activated potassium channel opener, or a pharmaceutically acceptable salt, solvate, hydrate, clathrate or prodrug thereof, can be readily determined by those skilled in the art. In general, a calcium activated potassium channel opener can be administered at a daily dose range of from about 0.01 mg to about 1,000 mg per day, from about 0.1 mg to about 500 mg per day, or from about 1 mg to about 100 mg per day, or from 2 mg to 50 mg per day.

The selected dosage level and frequency of administration of the pharmaceutical compositions of the invention will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

Stereomerically pure albuterol can be synthesized using any suitable methods known in the art. For example, stereomerically pure albuterol may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions, p. 268 (E. L. Eliel, Ed., Univ. of Notre Dame Press, Notre Dame, Ind., 1972), all of which are incorporated herein by reference.

Albuterol, or a pharmaceutically acceptable salt, solvate, stereoisomer, or prodrug thereof, and a calcium activated potassium channel opener, or a pharmaceutically acceptable slat, solvate, or prodrug thereof, can be administered to a patient via any suitable routes known in the art. Suitable routes of administration include, but are not limited to, oral, mucosal (e.g., nasal, sublingual, vaginal, buccal, or rectal), parenteral (e.g., subcutaneous, intravenous, bolus injection, intramuscular, or intraarterial), or transdermal administration. Albuterol, or a pharmaceutically acceptable salt, solvate, stereoisomer, or prodrug thereof, and a calcium activated potassium channel opener, or a pharmaceutically acceptable salt, solvate, or prodrug thereof, can be administered using the same route, or using different routes.

The actual dosage (quantity administered at a time) and the number of administrations per day will depend on the mode of administration, for example, by inhaler, nebulizer or oral administration. About 30 mcg to about 90 mcg of albuterol given by inhalation one or more times per day will be adequate in most individuals to produce the desired bronchodilation effect. For oral administration, e.g., tablet or syrup, a dose of about 1 mg to about 8 mg two to four times daily is administered to produce the desired effect. In addition, about 1 mg to 10 mg of andolast provides adequate administration dosage for inhalation, and about 1 mg to about 5 mg for intranasal application.

This invention also encompasses the administration of albuterol, or a pharmaceutically acceptable salt, solvate, stereoisomer, or prodrug thereof, and a calcium activated potassium channel opener, or a pharmaceutically acceptable salt, solvate, or prodrug thereof, optionally in combination with one or more further pharmacologically active agents. For example, an antiasthmatic drug such as theophylline or terbutaline, or an antihistamine or analgesic such as aspirin, acetaminophen or ibuprofen, can be given with or in close temporal proximity to administration of compounds of this invention. The administration may be sequential or concurrent.

4.2 Pharmaceutical Compositions

This invention encompasses pharmaceutical compositions comprising: albuterol, or a pharmaceutically acceptable salt, solvate, stereoisomer, or prodrug thereof; a calcium activated potassium channel opener, or a pharmaceutically acceptable salt, solvate, or prodrug thereof; and optionally a pharmaceutically acceptable carrier or excipient.

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic or hard gelatin capsules; cachets; troches; lozenges; dispersions; suppositories; ointments; cataplasms (poultices); pastes; powders; dry powders for inhalation; UDV nebulized solutions; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

In one embodiment, the dosage form is a UDV nebulized solution. The solution may be water, and the solution may further comprise a stabilizer. See, e.g., U.S. Pat. No. 6,667,344, which is incorporated in its entirety by reference. In another embodiment, the dosage form is dry powder for inhalation (e.g., capsules for aerosol). In another embodiment, the dosage form is an intranasal spray or solution.

The formulation should suit the mode of administration. For example, oral administration may require enteric coatings to protect the compounds of this invention from degradation within the gastrointestinal tract. In another example, the compounds of this invention may be administered in a liposomal formulation to shield the compounds from degradative enzymes, facilitate transport in circulatory system, and effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

The selected dosage level and frequency of administration of the pharmaceutical compositions of the invention will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

The pharmaceutical compositions of the invention may further comprise a pharmaceutically acceptable carrier. The term “pharmaceutically acceptable carrier” means one or more pharmaceutically acceptable excipients. Examples of such excipients are well known in the art and are listed in the USP(XXI)/NF (XVI), incorporated herein in its entirety by reference thereto, and include without limitation, binders, diluents, fillers, disintegrants, super disintegrants, lubricants, surfactants, antiadherents, stabilizers, and the like. The term “additives” is synonymous with the term “excipients” as used herein.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

Further, the term “pharmaceutically acceptable” excipient is employed to mean that there are no untoward chemical or physical incompatibilities between the active ingredients and any of the excipient components of a given dosage form. For example, an untoward chemical reaction is one wherein the potency of albuterol or a calcium activated potassium channel opener is detrimentally reduced or increased due to the addition of one or more excipients. Another example of an untoward chemical reaction is one wherein the taste of the dosage form becomes excessively sweet, sour or the like to the extent that the dosage form becomes unpalatable. Each excipient must be “acceptable” in the sense of being compatible with the other ingredients of the formulation and not injurious to the patient.

Physical incompatibility refers to incompatibility among the various components of the dosage form and any excipient(s) thereof. For example, the combination of the excipient(s) and the active ingredient(s) may form an excessively hygroscopic mixture or an excessively segregated mixture to the degree that the desired shape of the dosage form (e.g., tablet, troche etc.), its stability or the like cannot be sufficiently maintained to be able to administer the dosage form in compliance with a prescribed dosage regimen as desired.

It is noted that all excipients used in the pharmaceutical compositions or dosage forms made in accordance with the present invention preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable, provide bioavailability, stability, procedures for proper handling and storage and methods for their examination and formulas for their manufacture or preparation.

The stability of a pharmaceutical product may be defined as the capability of a particular formulation, in a specific container, to remain within its physical, chemical, microbiological, therapeutic and toxicological specification, although there are exceptions, and to maintain at least about 90% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredients and the like. Physical factors such as heat, light and moisture may initiate or accelerate chemical reactions.

4.2.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Large-scale production of pharmaceutical compositions or dosage forms in accordance with the present invention may require, in addition to the therapeutic drug ingredients, excipients or additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like.

Hence, unit dose forms or dosage formulations of a pharmaceutical composition of the present invention, such as a troche, a tablet or a capsule, may be formed by combining a desired amount of each of the active ingredients with one or more pharmaceutically compatible or acceptable excipients, as described below, in pharmaceutically compatible amounts to yield a unit dose dosage formulation the desired amount of each active ingredient. The dose form or dosage formulation may be formed by methods well known in the art.

Tablets are often a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

Tablets are typically made by molding, by compression or by generally accepted tablet forming methods. Accordingly, compressed tablets are usually prepared by large-scale production methods while molded tablets often involve small-scale operations. For example, there are three general methods of tablet preparation: (1) the wet-granulation method; (2) the dry-granulation method; and (3) direct compression. These methods are well known to those skilled in the art. See, Remington's Pharmaceutical Sciences, 16th and 18th Eds., Mack Publishing Co., Easton, Pa. (1980 and 1990). See, also, U.S. Pharmacopeia XXI, U.S. Pharmacopeial Convention, Inc., Rockville, Md. (1985).

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

Pharmaceutical compositions or unit dosage forms of the present invention in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In order for medicinal substances or therapeutic ingredients of the present invention, with or without excipients, to be made into solid dosage forms (e.g., tablets) with pressure, using available equipment, it is necessary that the material, either in crystalline or powdered form, possess a number of physical characteristics. These characteristics can include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drugs or therapeutic ingredients, tablets and similar dosage forms may contain a number of materials referred to as excipients or additives. These additives are classified according to the role they play in the formulation of the dosage form such as a tablet, a caplet, a capsule, a troche or the like. One group of additives include, but are not limited to, binders, diluents (fillers), disintegrants, lubricants, and surfactants. In one embodiment the diluent, binder, disintegrant, and lubricant are not the same.

A binder is used to provide a free-flowing powder from the mix of tablet ingredients so that the material will flow when used on a tablet machine. The binder also provides a cohesiveness to the tablet. Too little binder will give flow problems and yield tablets that do not maintain their integrity, while too much can adversely affect the release (dissolution rate) of the drugs or active ingredients from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with dosage formulations made in accordance with the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (povidone), methyl cellulose, pre-gelatinized starch, hydroxypropyl methyl cellulose, (e.g., Nos. 2208, 2906, 2910), microcrystalline cellulose or mixtures thereof. Suitable forms of microcrystalline cellulose can include, for example, the materials sold as AVICEL-PH-101, AVICEL-PH-103 and AVICEL-PH-105 (available from FMC Corporation, American Viscose Division, Avicel Sales, Marcus Hook, Pa., U.S.A.).

Fillers or diluents are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug(s) to the filler may occur and affect bioavailability. Consequently, a sufficient amount of filler should be used to achieve a desired dilution ratio without detrimentally affecting release of the drug ingredients from the dosage form containing the filler. Further, a filler that is physically and chemically compatible with the therapeutic ingredient(s) of the dosage form should be used. The amount of filler used varies upon the type of formulation and mode of administration, and is readily discernible to those of ordinary skill in the art. Examples of fillers include, but are not limited to, lactose, glucose, sucrose, fructose, talc, calcium carbonate (e.g., granules or powder), microcrystalline cellulose, powdered cellulose, dextrates, kaolin, mannitol, silicic acid, sorbitol, starch, pre-gelatinized starch, or mixtures thereof.

Disintegrants are used to cause the dose form (e.g., tablet) to disintegrate when exposed to an aqueous environment. Too much of a disintegrant will produce tablets which may disintegrate in the bottle due to atmospheric moisture. Too little may be insufficient for disintegration to occur and may thus alter the rate and extent of release of drug(s) or active ingredient(s) from the dosage form. Thus, a sufficient amount of disintegrant that is neither too little nor too much to detrimentally alter the release of the drug ingredients should be used to form the dosage forms made according to the present invention. The amount of disintegrant used varies based upon the type of formulation and mode of administration, and is readily discernible to the skilled artisan. Examples of disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof.

When a dose form that dissolves fairly rapidly upon administration to the subject, e.g., in the subject's stomach is desired, a super disintegrant can be used, such as, but not limited to, croscarmellose sodium or sodium starch glycolate. The term “super disintegrant,” as used herein, means a disintegrant that results in rapid disintegration of drug or active ingredient in the stomach after oral administration. Use of a super disintegrant can facilitate the rapid absorption of drug or active ingredient(s) which may result in a more rapid onset of action.

Adhesion of the dosage form ingredients to the punches of the manufacturing machine (e.g., a tableting machine) must be avoided. For example, when drug accumulates on the punch surfaces, it causes the tablet surface to become pitted and therefore unacceptable. Also, sticking of drug or excipients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing or by the use of lubricants, e.g., magnesium stearate. However, selection of a drug salt with good anti-adhesion properties can also minimize these problems.

As noted, the lubricant is used to enhance the flow of the tableting powder mix to the tablet machine and to prevent sticking of the tablet in the die after the tablet is compressed. Too little lubricant will not permit satisfactory tablets to be made and too much may produce a tablet with a water-impervious hydrophobic coating, which can form because lubricants are usually hydrophobic materials such as stearic acid, magnesium stearate, calcium stearate and the like. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Example of suitable lubricants for use with the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W.R. Grace Co. of Baltimore Md.), a coagulated aerosol of synthetic silica (marketed by Deaussa Co. of Plano, Tex.), CAB-O-SIL (a pyrogenic silicon dioxide product sold by Cabot Co. of Boston, Mass.) or mixtures thereof.

Surfactants are used in dosage forms to improve the wetting characteristics and/or to enhance dissolution, and are particularly useful in pharmaceutical compositions or dosage forms containing poorly soluble or insoluble drug(s) or active ingredients. Examples of surfactants include, but are not limited to, polyoxyethylene sorbitan fatty acid esters, such as those commercially available as TWEENs (e.g. Tween 20 and Tween 80), polyethylene glycols, polyoxyethylene stearates, polyvinyl alcohol, polyvinylpyrrolidone, poly(oxyethylene)/poly(oxypropylene) block co-polymers such as poloxamers (e.g., commercially available as PLURONICs), and tetrafunctional block copolymers derived from sequential addition of propylene oxide and ethylene oxide to ethylenediamine, such as polyxamines (e.g., commercially as TETRONICs (BASF)), dextran, lecithin, dialkylesters of sodium sulfosuccinic acid, such as Aerosol OT, sodium lauryl sulfate, alkyl aryl polyether sulfonates or alcohols, such as TRITON X-200 or tyloxapol, p-isononylphenoxypoly (glycidol) (e.g. Olin-10G or Surfactant 10-G (Olin Chemicals), or mixtures thereof. Other pharmaceutically acceptable surfactants are well known in the art, and are described in detail in the Handbook of Pharmaceutical Excipients.

Other classes of additives for use with the pharmaceutical compositions or dosage forms of the present invention include, but are not limited to, anti-caking or antiadherent agents, antimicrobial preservatives, coating agents, colorants, desiccants, flavors and perfumes, plasticizers, viscosity increasing agents, sweeteners, buffering agents, humectants and the like.

Examples of anti-caking agents include, but are not limited to, calcium silicate, magnesium silicate, silicon dioxide, colloidal silicon dioxide, talc, or mixtures thereof.

Examples of antimicrobial preservatives include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol, or mixtures thereof.

Examples of colorants for use with the present invention include, but are not limited to, pharmaceutically acceptable dyes and lakes, caramel, red ferric oxide, yellow ferric oxide or mixtures thereof. Examples of desiccants include, but are not limited to, calcium chloride, calcium sulfate, silica gel or mixtures thereof.

Flavors that may be used include, but are not limited to, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Examples of sweetening agents include, but are not limited to, aspartame, dextrates, mannitol, saccharin, saccharin calcium, saccharin sodium, sorbitol, sorbitol solution, or mixtures thereof.

Exemplary plasticizers for use with the present invention include, but are not limited to, castor oil, diacetylated monoglycerides, diethyl phthalate, glycerin, mono- and di-acetylated monoglycerides, polyethylene glycol, propylene glycol, and triacetin or mixtures thereof. Suitable viscosity increasing agents include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Buffering agents that may be used in the present invention include, but are not limited to, magnesium hydroxide, aluminum hydroxide and the like, or mixtures thereof. Examples of humectants include, but are not limited to, glycerol, other humectants or mixtures thereof.

The dosage forms of the present invention may further include one or more of the following: (1) dissolution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g., ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

Dosage forms of the present invention, such as a tablet or caplet, may optionally be coated. Inert coating agents typically comprise an inert film-forming agent dispersed in a suitable solvent, and may further comprise other pharmaceutically acceptable adjuvants, such as colorants and plasticizers. Suitable inert coating agents, and methods for coating, are well known in the art, including without limitation aqueous or non-aqueous film coating techniques or microencapsulation. Examples of film-forming or coating agents include, but are not limited to, gelatin, pharmaceutical glaze, shellac, sucrose, titanium dioxide, carnauba wax, microcrystalline wax, celluloses, such as methylcellulose, hydroxymethyl cellulose, carboxymethylcellulose, cellulose acetate phthalate, hydroxypropyl methylcellulose (e.g., Nos.: 2208, 2906, 2910), hydroxypropyl cellulose, hydroxypropyl methyl cellulose phthalate (e.g., Nos.: 200731, 220824), hydroxyethylcellulose, methylhydroxyethylcellulose, ethylcellulose which may optionally be cross-linked, and sodium carboxymethyl cellulose; vinyls, such as polyvinyl pyrrolidione, polyvinyl acetate phthalate,; glycols, such as polyethylene glycols; acrylics, such as dimethylaminoethyl methacrylate-methacrylate acid ester copolymer, and ethylacrylate-methylmethacrylate copolymer; and other carbohydrate polymers, such as maltodextrins, and polydextrose, or mixtures thereof. The amount of coating agent and the carrier vehicle (aqueous or non-aqueous) used varies upon the type of formulation and mode of administration, and is readily discernible to those of ordinary skill in the art.

A coating of a film forming polymer may optionally be applied to a tablet or caplet (e.g., a capsule shaped tablet) in accordance with the present invention by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution.

The coating solution may be sprayed by using positive pneumatic displacement or peristaltic pump systems in a continuous or intermittent spray-dry cycle. The particular type of spray application is selected depending upon the drying efficiency of the coating pan. In most cases, the coating material is sprayed until the tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use in coating a final dosage form, such as tablets include, for example, methylcellulose, hydroxypropyl methyl cellulose (PHARMACOAT 606 6 cps), polyvinylpyrrolidone (povidone), ethylcellulose (ETHOCEL 10 cps), various derivatives of methacrylic acids and methacrylic acid esters, cellulose acetate phthalate or mixtures thereof.

The method of preparation and the excipients or additives to be incorporated into dosage form (such as a tablet or caplet) are selected in order to give the tablet formulation the desirable physical characteristics while allowing for ease of manufacture (e.g., the rapid compression of tablets). After manufacture, the dose form preferably should have a number of additional attributes, for example, for tablets, such attributes include appearance, hardness, disintegration ability and uniformity, which are influenced both by the method of preparation and by the additives present in the tablet formulation.

Further, it is noted that tablets or other dosage forms of the pharmaceutical compositions of the invention should retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredients should not change appreciably with time.

The tablets, and other dosage forms of the pharmaceutical compositions of the present invention, such as dragees, capsules, pills and granules, may optionally be scored or prepared with coatings and shells, such as enteric coatings and other coatings well known in the pharmaceutical formulating art.

4.2.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds of this invention) disclosed herein can also be incorporated into the parenteral dosage forms of the invention.

4.2.3 Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.2.4 Compositions with Enhanced Stability

The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

4.2.5 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the compounds of this invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.2.6 Kits

In some cases, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a single unit dosage form of the compounds of this invention, or a pharmaceutically acceptable salt, hydrate, prodrug, solvate, or clathrate thereof, and a single unit dosage form of another agent that may be used in combination with the compounds of this invention. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The invention is further defined by reference to the following non-limiting examples. It will be apparent to those skilled in the art that many modifications, both to materials and methods, can be practiced without departing from the spirit and scope of this invention.

4.2.7 Dry Powder for Inhalation

In a further embodiment, the pharmaceutical composition is in the form of a dry powder suitable for inhalation or insufflation. To be suitable as a dry powder for inhalation (DPI), the active compounds or compounds must be inhalable. In order to pass into the lungs, the particles should have a size of from about 1 to 10 μm. In one embodiment, the particle size is preferably from about 1 to about 5 μm.

Such microfine particles may be obtained, for example, by micronization, controlled precipitation from suitable solvents or by spray drying if the process conditions are suitably selected, controlled and carried out. Since microfine particles have strong tendencies to adhere which may lead to poor flow properties and difficulty in handling, the particles are typically mixed with pharmacologically inactive excipients to obtain a dosable unit amount as disclosed in U.S. Pat. Nos. 5,976,576 and 6,645,466, both incorporated herein by reference in their entirety.

According to another aspect of the invention, therefore, the present invention comprises albuterol, or a pharmaceutically acceptable salt, solvate, stereoisomer, or prodrug thereof, and a calcium activated potassium channel opener, as described herein, together with one or more suitable pharmaceutical excipients in a form suitable for DPI. In one embodiment the calcium activated potassium channel opener is andolast. Suitable excipients include, but are not limited to, carriers typically used in dry powder formulations, for example, mono- or disaccharides, such as, but not limited to, glucose, lactose, lactose monohydrate, sucrose, trehalose, sugar alcohols, such as mannitol or xylitol, polylactic acid or cyclodextrin, amino acids and proteins, and/or in the form of their pharmaceutically acceptable esters, acetals, or salts(where such derivatives exist). In one embodiment, the carrier is preferably lactose, more preferably lactose monohydrate.

In addition, since andolast has a bitter taste, a sweetener may also be included in the pharmaceutical formulation. The sweetener may be selected from carbohydrates such as, for example, sucrose, fructose, glucose, mannitol, or aspartame, sodium cyclamate, saccharin, sodium saccharin and the like. The pharmaceutical composition may comprise a sweetener or sweeteners which may be present in the composition in a ratio by weight (weight/weight) of between about 1 and 30%, preferably between 10 and 20% with reference to the weight of the active ingredient.

The compositions may also comprise a flavoring, such as menthol or peppermint oil, which functions as a taste-mask as well as a means to increase the respirable fraction of the drug as disclosed in, for example, U.S. Pat. No. 5,976,576, referred to previously and incorporated by reference.

In one embodiment, the DPI formulation can be prepared by dissolving the flavoring in a volatile, non-aqueous solvent such as, for example, ethyl ether or preferably methylene chloride and mixing the solution with the active ingredients in the ratios conventionally known in the art, as well as those described herein. In this manner, after evaporation of the solvent, a homogeneous, free-flowing, non-sticky mixture is obtained, and the tendency of the particles to form agglomerates is greatly reduced.

In one embodiment, in order to increase the fluidity of the pharmaceutical composition within the reservoir of an inhaler device, the mixture can be transformed into pellets by tumbling it with conventional techniques and sieved through stainless steel sieves to provide pellets having a size between 100-1000 micrometers.

The dry powder composition may be presented in unit dosage form, for example, capsules or cartridge of, e.g., gelatin, or blister packs from which the powder may be administered with the aid of an inhaler or insufflator. The dry powder composition may be presented in multi dose form metered with the aid of an inhaler or insufflator.

Dry powder formulations may also be administered using multidose dry powder inhalers. The present invention also provides a multidose dry powder inhaler, comprising a dry powder reservoir containing a dry powder aerosol formulation as described herein, and a metering chamber. Administration could be achieved with a metered dose inhaler (MDI) or a pressurized metered dose inhaler (PMDI).

In a further embodiment, in order to increase the fluidity of the pharmaceutical composition within the reservoir of an inhaler device, the mixture can be transformed into pellets by tumbling it with conventional techniques and sieved through stainless steel sieves to provide pellets having a size between 100-1000 micrometers.

### 5. EXAMPLES

The following examples illustrate specific pharmaceutical compositions of the invention.

### 5.1 Example 1

| | | |
|---|---|---|
| Albuterol | 1 | mg |
| Andolast | 2 | mg |
| Lactose monohydrate | 0.2-10 | mg |

### 5.2 Example 2

| | | |
|---|---|---|
| Albuterol | 4 | mg |
| Andolast | 4 | mg |
| Lactose monohydrate | 0.2-10 | mg |

### 5.3 Example 3

| | | |
|---|---|---|
| Albuterol | 2 | mg |
| Andolast | 8 | mg |
| Lactose monohydrate | 0.2-10 | mg |

### 5.4 Example 4

| | | |
|---|---|---|
| Albuterol | 4 | mg |
| DHS-I | 8 | mg |
| Lactose monohydrate | 0.2-10 | mg |

### 5.5 Example 5

| | | |
|---|---|---|
| Albuterol | 2 | mg |
| DHS-I | 500 | μg |
| Lactose monohydrate | 0.2-5 | mg |

### 5.6 Example 6

| | | |
|---|---|---|
| Albuterol | 4 | mg |
| DHS-I | 5 | mg |
| Lactose monohydrate | 0.2-10 | mg |

### 5.7 Example 7

| | | |
|---|---|---|
| R(−)-Albuterol | 1 | mg |
| Andolast | 2 | mg |
| Lactose monohydrate | 0.2-10 | mg |

### 5.8 Example 8

| | | |
|---|---|---|
| R(−)-Albuterol | 4 | mg |
| Andolast | 4 | mg |
| Lactose monohydrate | 0.2-10 | mg |

### 5.9 Example 9

| | | |
|---|---|---|
| R(−)-Albuterol | 2 | mg |
| Andolast | 8 | mg |
| Lactose monohydrate | 0.2-10 | mg |

### 5.10 Example 10

| | | |
|---|---|---|
| R(−)-Albuterol | 4 | mg |
| DHS-I | 8 | mg |
| Lactose monohydrate | 0.2-10 | mg |

### 5.11 Example 11

| | | |
|---|---|---|
| R(−)-Albuterol | 2 | mg |
| DHS-I | 500 | μg |
| Lactose monohydrate | 0.2-5 | mg |

### 5.12 Example 12

| | | |
|---|---|---|
| R(−)-Albuterol | 4 | mg |
| DHS-I | 5 | mg |
| Lactose monohydrate | 0.2-10 | mg |

### 5.13 Example 13

Following is a formulation suitable for UDV with 1% andolast and 0.63 mg of levalbuterol HCl.

| Component | Weight | |
|---|---|---|
| Levalbuterol HCl | 0.726 | g |
| Andolast | 30 | g |
| NaCl | 20.1 | g |
| QS to 3000 grams | | |

Add levalbuterol HCl, andolast and NaCl to 500 grams of water. QS to 3000 grams with water.

All of the patents, patent applications and publications referred to in this application are incorporated herein in their entireties. Moreover, citation or identification of any reference in this application is not an admission that such reference is available as prior art to this invention. The full scope of the invention is better understood with reference to the appended claims.

1. A method of treating a pulmonary or respiratory disease or disorder which comprises administering to a patient in need of such treatment a therapeutically effective amount of albuterol, or a pharmaceutically acceptable salt or solvate thereof, and a therapeutically effective amount of a calcium activated potassium channel opener, or a pharmaceutically acceptable salt or solvate thereof.

2-3. (canceled)

4. The method of claim 1, wherein albuterol is racemic.

5. The method of claim 1, wherein albuterol is stereomerically pure.

6. The method of claim 5, wherein the stereomerically pure albuterol is R(−)-albuterol.

7. The method of claim 1, wherein albuterol, or a pharmaceutically acceptable salt or solvate thereof, and the calcium activated potassium channel opener, or a pharmaceutically acceptable salt or solvate thereof, are concurrently administered.

8. The method of claim 1, wherein albuterol, or a pharmaceutically acceptable salt or solvate thereof, and the calcium activated potassium channel opener, or a pharmaceutically acceptable salt or solvate thereof, are sequentially administered.

9. The method of claim 1, wherein the calcium activated potassium channel opener is andolast, DHS-I, soyasaponin I, soyasaponin III, NS 004, NS 1619, or BRL 55834.

10. The method of claim 9, wherein the calcium activated potassium channel opener is andolast.

11. The method of claim 9, wherein the calcium activated potassium channel opener is DHS-I.

12. The method of claim 1, wherein the pulmonary disease or disorder is respiratory failure; adult respiratory distress syndrome; cystic fibrosis; a chronic obstructive airway disorder; acute bronchitis; chronic bronchitis; bronchiolitis; emphysema; reversible obstructive airway disease; nocturnal asthma; exercise induced bronchospasm; bronchiectasis; atelectasis; pulmonary embolism; pneumonia; lung abscess; hypersensitivity of the lung; or Goodpasture's syndrome.

13. The method of claim 1, wherein the treatment is long-term maintenance treatment of asthma.

14-16. (canceled)

17. The method of claim 12, wherein the hypersensitivity of the lung is hypersensitivity pneumonitis, eosinophilic pneumonias or allergic bronchopulmonary aspergillosis.

18. The method of claim 12, wherein the pulmonary disease or disorder is a chronic obstructive airway disorder.

19. The method of claim 18, wherein the chronic obstructive airway disorder is asthma or chronic obstructive pulmonary disease.

20. The method of claim 12, wherein the pulmonary disease or disorder is asthma.

21. A pharmaceutical composition comprising: albuterol, or a pharmaceutically acceptable salt or solvate thereof, a calcium activated potassium channel opener, or a pharmaceutically acceptable salt or solvate thereof.

22. The composition of claim 21, wherein albuterol is racemic.

23. The composition of claim 21, wherein albuterol is stereomerically pure.

24. The composition of claim 23, wherein the stereomerically pure albuterol is R(−)-albuterol.

25. The composition of claim 21, wherein the calcium activated potassium channel opener is andolast, DHS-I, soyasaponin I, soyasaponin III, NS 004, NS 1619, or BRL 55834.

26. The composition of claim 25, wherein the calcium activated potassium channel opener is andolast.

27. The composition of claim 25, wherein the calcium activated potassium channel opener is DHS-I.

28. A single dosage form comprising albuterol, or a pharmaceutically acceptable salt or solvate thereof, and a calcium activated potassium channel opener, or a pharmaceutically acceptable salt or solvate thereof.

29. The single dosage form of claim 28, wherein albuterol is racemic.

30. The single dosage form of claim 28, wherein albuterol is stereomerically pure.

31. The single dosage form of claim 30, wherein the stereomerically pure albuterol is R(−)-albuterol.

32. The single dosage form of claim 28, wherein the calcium activated potassium channel opener is andolast, DHS-I, soyasaponin I, soyasaponin III, NS 004, NS 1619, or BRL 55834.

33. The single dosage form of claim 32, wherein the calcium activated potassium channel opener is andolast.

34. The single dosage form of claim 32, wherein the calcium activated potassium channel opener is DHS-I.

35. The dosage form of claim 28, wherein the dosage form is suitable for oral, parenteral, topical, or mucosal administration.

36. The dosage form of claim 35, wherein the dosage form is suitable for oral or mucosal administration.

37. The dosage form of claim 28, which is suitable for administration as a UDV nebulized solution.

38. The dosage form of claim 28, which is suitable for administration as an intranasal spray or intranasal liquid solution.

39. The dosage form of claim 28, which is suitable for administration as dry powder for inhalation.

40. The dosage form of claim 39, wherein the dry powder for inhalation is administered using a metered dose inhaler or a pressurized metered dose inhaler.
